(19) United States
(12) Patent Application Publication
(10) Pub. No.: US 2007/0244053 A1
(43) Pub. Date: Oct. 18, 2007

(54) Use of a CD40:CD154 binding interruptor to prevent counter-adaptive immune responses, particularly graft rejection

(76) Inventors: Allan D. Kirk, Potomac, MD (US); David M. Harlan, Potomac, MD (US); David W. Thomas, Wellesley, MA (US); Michael Kauffman, Newton, MA (US); Linda Burkly, West Newton, MA (US)

Correspondence Address: FISH & NEAVE IP GROUP;ROPES & GRAY LLP, 1211 AVENUE OF THE AMERICAS, NEW YORK, NY, 10036-8704, US

(21) Appl. No.: 11/638,218

(22) Filed: Dec. 12, 2006

Related U.S. Application Data
(63) Continuation of application No. 10/120,272, filed on Apr. 9, 2002, now abandoned.
Application No. 10/120,272 is a continuation of application No. 09/442,012, filed on Nov. 17, 1999, now abandoned.
Application No. 09/442,012 is a continuation-in-part of application No. PCT/US98/10075, filed on May 15, 1998.
(60) Provisional application No. 60/085,145, filed on May 12, 1998.
(60) Provisional application No. 60/049,389, filed on Jun. 11, 1997.
(60) Provisional application No. 60/046,791, filed on May 17, 1997.

(51) Int. Cl. A61K 39/395; A61K 38/00; C07K 14/705; C07K 16/28; A61K 38/17

(52) U.S. Cl. 514/13; 530/388.22

(57) ABSTRACT

Compositions and methods disclosed herein capitalize on the discovery that rejection of a tissue graft can be inhibited using a CD40:CD154 binding interrupter, either alone or in combination with another immunomodulator or immunosuppressor. An advantageous, synergistic combination includes a CD40:CD154 binding interrupter and a CD28 signaling interrupter. An exemplary CD40:CD154 binding interrupter is an anti-CD154 monoclonal antibody, such as an antibody having the antigen-specific binding characteristics of the 5c8 monoclonal antibody. An exemplary CD28 signaling interrupter is a CTLA4-Ig fusion protein. The disclosed compositions and methods unexpectedly can be used to prolong survival of grafted tissue in a recipient host, to reverse acute graft rejection, and to attenuate immunological consequences of the failure of grafted tissue.

### RELATED APPLICATIONS

This is a continuation-in-part of PCT application number PCT/US98/10075, filed May 15, 1998, which is a continuation-in-part of U.S. provisional application No. 60/085,145, filed May 12, 1998, a continuation-in-part of U.S. provisional application No. 60/046,791, filed May 17, 1997, and a continuation-in-part of U.S. provisional application No. 60/049,389, filed Jun. 11, 1997. The teachings of all four earlier-filed patent applications are incorporated. herein by reference.

### TECHNICAL FIELD OF THE INVENTION

The present invention relates generally to the suppression of unwanted immune responses, particularly of counter-adaptive T-lymphocyte mediated immune responses. This invention relates in particular to the prevention, treatment, suppression or reversal of immune-system driven rejection of grafted tissue, including skin, or a grafted organ or a portion thereof, or a body part, such as a joint or a finger, with multiple tissue types in a recipient host. According to a preferred embodiment of this invention, such effects are achieved using a CD40:CD154 binding interrupter.

### BACKGROUND OF THE INVENTION

Organ transplantation between genetically non-identical individuals invariably results in immunological rejection of the organ through T cell dependent mechanisms, unless the rejection process is bridled by drugs that suppress T cell function. Several United States patents disclose the use of such immunosuppressant drugs for inhibiting graft rejection, including U.S. Pat. Nos. 5,104,858; 5,008,246; and, 5,068,323. Other conventional agents are described in Suthanthiran et al. (1994), 331 New Eng. Med. J. 365-376. Both calcineurin phosphatase inhibitors and glucocorticosteroids are used clinically, and both prevent the T cell mediated release of activating cytokines, particularly IL-2. However, therapy with these types of conventional agents remains imperfect. Such agents act by impairing signaling through the T cell antigen receptor (TCR), the sole mediator of T cell antigen specificity, and act on all T cells indiscriminately. In addition, the effect of these drugs is not lasting,.such that cessation of treatment generally results in graft loss. Thus, in order to maintain viable, functional integration of the graft, transplant recipients must suffer the consequences of long-term, non-specific immunosuppression. These consequences include an increased risk of infection and malignancy, as well as significant expense and toxicity.

There is accordingly a need for improved or more effective immunosuppressive or immunomodulatory treatments for graft recipients. In particular, there is a need for treatments that do not promote pan-T cell immunosuppression, i.e., treatments that do not leave the recipient vulnerable to malignancies or opportunistic infection. More pointedly, there is a need for treatments that have less toxicity than conventional therapeutic agents. Similarly, there is a need for treatments that promote lasting functional integration of the graft, i.e., integration that persists beyond termination of the course of treatment.

### SUMMARY OF THE INVENTION

It is an object of this invention to provide an immunomodulatory agent that mitigates counter-adaptive T cell responses without the need for pan-T cell immunosuppression. Another object is to provide an immunomodulatory agent that promotes functional integration of a tissue graft in a recipient host. Another object is to provide an immunomodulatory agent that inhibits immunological rejection of grafted tissue. A further object is to provide an immunomodulatory agent that interrupts delivery of a costimulatory signal to activated T cells. A particular object is to provide a CD40:CD154 binding interrupter for use in therapy, particularly for use in therapy to mitigate or delay immunological rejection of grafted tissue. Another particular object is to provide a therapeutic composition and treatment regime for mitigating counter-adaptive T cell mediated immune responses, based on the use of a CD40:CD154 binding interrupter in combination with another immunosuppressant or immunomodulator. Thus, a specific object of the invention is to provide a therapeutic composition and treatment regime based on the use of a CD40:CD154 binding interruptor in combination with an agent that blocks costimulation via CD28. A more general object of the invention is to provide a therapeutic composition and treatment regime for inhibiting, mitigating, attenuating, delaying or reversing failure or acute rejection of grafted tissue or delaying chronic rejection of grafted tissue. Another general object of the invention is to improve the availability of tissue grafts, by providing immunomodulatory compositions that allow functional. integration of allogeneic or xenogeneic tissue into a recipient host. -A still further general object is to prevent, mitigate, attenuate or treat disease states resulting from a counter-adaptive immune response, including T-lymphocyte mediated autoimmune illnesses (e.g., insulin dependent diabetes mellitus, multiple sclerosis and the like), as well as allergic illnesses. The present invention rests on the discovery that use of a CD40:CD154 binding interrupter, alone or in combination with another immunomodulatory agent, attenuates, suppresses, prevents, delays or reverses counter-adaptive immune system rejection of grafted tissue in a recipient host, without the need for pan-suppression of the recipient's immune system.

The invention accordingly provides methods and compositions for immunomodulatory therapy for recipients of grafted tissue. A first method inhibits rejection of a tissue graft by a graft recipient, by treating the graft recipient with a CD40:CD154 (CD40L) binding interrupter. Such a binding interrupter is any agent that interrupts the binding of a costimulatory molecule (here, CD40 ligand, also referred to herein as the 5c8 antigen, T-BAM, CD40L, CD154, and also referred to in the art as gp39) to its counter or cognate receptor (here, CD40). Preferably, the binding interruptor is an anti-CD40L compound, by which is meant a compound that binds to CD40L (CD154) and thereby interferes with or disrupts the ability of CD40L to bind to CD40. In some embodiments, the binding interrupter may cause depletion in vivo of cells expressing CD40L. An exemplary anti-CD40L compound is a monoclonal antibody, particularly an antibody having the antigen-specific binding characteristics of the 5c8 monoclonal antibody disclosed in U.S. No. 5,474,771, the teachings of which are incorporated herein by reference.

A second method prolongs survival of a tissue graft in a graft recipient, by treating the graft recipient with a CD40:CD154 binding interrupter, preferably an anti-CD40L monoclonal antibody. A third method attenuates immunological complications of failure of grafted by treating a graft recipient with a CD40:CD154 binding interrupter, preferably an anti-CD40L monoclonal antibody. That is, the method inhibits, suppresses, mitigates or detectably decreases such immunological complications. In particular, the method avoids or mitigates complications such as interstitial fibrosis, chronic-graft atherosclerosis, vasculitis and the like.

The foregoing methods are effective for treatments of acute and/or chronic rejection of grafted tissue and can be used prophylactically, for post-operative treatment, or for reversing or suppressing graft rejection at any time during the recipient's lifetime. An exemplary method involves administration of a CD40:CD154 binding interrupter on postoperative days, such as days 0, 2, 4, 6, 8, 12, 16 and 28. More generally, the methods described herein involve administration of the binding interrupter at desired intervals (daily, twice weekly, weekly or biweekly) over at least a two-or three-week period. The administration schedule is adjusted as needed to produce a detectable decrease in indicia of counter-adaptive immune responses, particularly indicia of graft rejection. The present treatment regime can be repeated in the event of a subsequent episode of graft rejection. Also, the tissue may be exposed to a CD40:CD154 binding interrupter prior to transplant. In embodiments wherein the binding interrupter is an anti-CD40L monoclonal antibody, the interrupter is administered at doses between about 0.05 mg/kg body weight and about 70 mg/kg body weight, more preferably, between about 1 and about 50 mg/kg, still more preferably, between about 1 and about 20 mg/kg body weight.

For treatment, the CD40:CD154 binding interrupter can be formulated in a therapeutic composition which includes a therapeutically effective amount of the binding interrupter dispersed in a pharmaceutically acceptable carrier. In some embodiments, the therapeutic composition can also include a therapeutically effective amount of another immunosuppressive or immunomodulatory compound, including without limitation: an agent that interrupts T cell costimulatory signaling via CD28 (e.g., CTLA4-Ig); an agent that interrupts calcineurin signaling (e.g., cyclosporine, a macrolide such as tacrolimus,-formerly known as FK506); a corticosteroid; or an antiproliferative agent (e.g., azathioprine or mycophenolate mofetil (MMF)). Other therapeutically effective compounds suitable for use with the present CD40:CD154 binding interrupter include sirolimus (formerly known as rapamycin); mizoribine, deoxyspergualin, brequinar sodium, leflunomide, azaspirane, cyclophosphamide and the like.

The methods and compositions of the invention are suitable for use with all types of graft procedures. Thus, the invention is suitable for use where the graft recipient (recipient host) is a mammal, preferably a primate, most preferably a human. The graft donor may be a non-syngeneic member of the same phylogenetic species as the graft recipient (i.e., an allogeneic donor, providing allograft tissue), or a member of a distinct phylogenetic species (i.e., a xenogeneic donor, providing xenograft tissue). If a xenogeneic donor is used as the graft tissue source, preferably the donor is relatively MHC-compatible with the recipient host; for example, a baboon or chimpanzee would be preferred as a donor for grafting tissue into a human. The invention can be used to promote engraftment of any body tissue, including skin, or organ type, regardless of whether the donor (graft) tissue be an entire organ, section or portion of an organ or tissue, a body part with multiple tissue types or isolated cells. Non-limiting examples of suitable tissues include renal, hepatic, cardiac, pancreatic (e.g., islet), skin, vascular, nerve, bone, cartilage and like mammalian body tissues.

As disclosed herein, the principles of the present invention have been validated by testing in a relevant preclinical model. An exemplary CD40:CD154 binding interrupter (the anti-CD40L monoclonal antibody 5c8) has been tested alone and in combination with other exemplary immunomodulators (the CD28 binding interrupter CTLA4-Ig; mycophenolate mofetil; corticosteroids; tacrolimus), on rhesus peripheral blood leukocytes in vitro, in rhesus macaques transplanted with skin allografts, and in rhesus monkeys transplanted with primarily vascularized renal allografts.

The foregoing and other objects, features and advantages of the present invention, as well as the invention itself, will be more fully understood from the following description of preferred embodiments.

### DETAILED DESCRIPTION OF THE INVENTION

Data establishing that T cell activation requires both TCR mediated signals and simultaneously delivered costimulatory signals have accumulated over the past twenty years. For example, antibody production by B lymphocytes in response to protein antigens requires a specific, costimulatory interaction with T lymphocytes. This B cell/T cell interaction is mediated through several receptor-ligand binding events in addition to engagement of the TCR. These additional binding events include the binding of CD40 on B cells to CD154 (CD40L) on T cells. Human CD40 is a 50 kD cell surface protein expressed on mature B cells, as well as macrophages, dendritic cells, fibroblasts and activated endothelial cells. CD40 belongs to a class of receptors involved in programmed cell death, including Fas/CD95 and the tumor necrosis factor (TNF) alpha receptor. Human CD154 (CD40L) is a 32 kD type II membrane glycoprotein with homology to TNF alpha that is transiently expressed primarily on activated T cells. CD40:CD154 binding. has been shown to be required for T cell-dependent antibody responses. In particular, CD40:CD154 binding provides anti-apoptotic and/or lymphokine stimulatory signals.

Another important costimulatory signal is produced by the binding of CD28 on T cells to its counter receptor CD80 (B7-1) or CD86 (B7-2) on antigen presenting cells (APCs) and perhaps also on parenchymal cells. Significantly, CD80 and/or CD86 expression is upregulated by signals initiated on the binding of CD40 to CD154. Further studies have shown that the T cell molecule CTLA4 (CD152) appears to down-regulate costimulation and TCR mediated activation, at least in part by competing with CD28 for CD80/CD86, and by delivering a unique negative signal to the TCR signal transduction complex.

The importance of CD40:CD154 binding in promoting-T cell dependent biological responses is underscored by the. development of X-linked hyper-IgM syndrome (X-HIGM) in humans lacking functional CD154. These individuals have normal or high IgM levels, but fail to produce IgG, IgA or IgE antibodies. Affected individuals suffer from recurrent, sometimes severe, bacterial infection (most commonly with Streptococcus pneumoniae, Pneumocystis carinii and Hemophilus influenzae) and certain unusual parasitic infections, as well as an increased incidence of lymphomas and abdominal cancers. These clinical manifestations of disease can be managed through intravenous immunoglobulin replacement therapy.

The effects of X-HIGM are simulated in animals rendered nullizygous for the gene encoding CD154 (knockout animals). Studies with nullizygotes have confirmed that, while B cells can produce IgM in the absence of CD40L:CD154 binding, they are unable to undergo isotype switching, or to survive normally and undergo affinity maturation. In the absence of a functional CD40:CD154 interaction, spleen and lymph node germinal centers do not develop properly, and the development of memory B cells is impaired. These defects contribute to a severe reduction or absence of a secondary (mature) antibody response.

Individuals with X-HIGM and CD154nullizygotes also have defects in cellular immunity. These defects are manifested by an increased incidence of Pneumocvstis carinii, Histoplasma capsulatum, Cryptococcus neoformans infection, as well as chronic Giardia lambli infection. Murine nullizygotes are deficient in their ability to fight Leishmaniainfection. Many of these cell-mediated defects are reversible by administration of IL-12 or IFN-gamma. These data substantiate the view that-CD40:CD154 binding promotes the development of Type I T-helper cell responses. Further support is derived from the observation that macrophage activation is defective in CD154-deficient settings, and that administration of anti-CD40L antibodies to mice diminished their ability to clear Pneumocystis infection. Blockade of CD40:CD154 binding appears to reduce the ability of macrophages to produce nitric oxide, which mediates many of the macrophage's pro-inflammatory activities. It should be noted, however, that mammals (including humans) who lack functional CD154 do not develop significant incidences of viral infection.

A number of preclinical studies have established that agents capable of interrupting CD40:CD154 binding have promise as immunomodulating agents. In murine systems, antibodies to CD154 block primary and secondary immune responses to exogenous antigens, both in vitro and in vivo. Antibodies to CD154 cause a reduction in germinal centers in mice and monkeys, consistent with data on CD154immunodeficiency. Administration of three doses of anti-CD154 antibody to lupus-prone mice, aged three months, substantially reduced titers against double-stranded DNA and nucleosomes, delayed the development of severe nephritis, and reduced mortality. Moreover, administration of anti-CD154 antibodies to mice aged five to seven months with severe nephritis was shown to stabilize or even reverse renal disease. Anti-CD154 antibodies given concomitantly with small resting allogeneic lymphocytes permitted unlimited survival of mouse pancreatic islet allografts. In other animal models, interference with CD40:CD154 binding has been demonstrated to reduce symptoms of autoimmune disease (e.g., multiple sclerosis, rheumatoid arthritis, inflammatory bowel disease), graft rejection (cardiac allograft, graft-versus-host disease), and mercuric chloride induced glomerulonephritis, which is mediated by both humoral and cellular mechanisms.

Additional studies in rodents have shown that T cell activation can be blocked, and rodent allograft survival prolonged, by interfering with the binding of CD80/CD86 to its T cell counter receptors, CD28 and CTLA4. These studies involved the use of the CD80/CD86 specific fusion protein, CTLA4-Ig, as a CD28 signaling interruptor. Others have demonstrated that CD80/CD86up-regulation can be prevented by use of a CD40:CD154 binding interrupter (e.g., the monoclonal antibody MR1, which specifically binds mouse CD40L). Both classes of immunomodulatory agents appear to be dependent on TCR engagement for their effectiveness. Thus, such agents offer the capacity to modulate the specificity of T cell dependent biological processes, rather than depending on pan T cell immunosuppression. Studies involving the use of such agents in vivo in rodent models of graft rejection have produced dramatic results, including the acceptance of fully mismatched skin grafts when used in conjunction with CTLA4-Ig, a result not obtainable with currently available immunosuppression.

It is noteworthy, however, that all previously reported studies of long-term graft survival in rodents have failed, or have been associated with unacceptable toxicity, when tested in other mammals, particularly primates.

Disclosed proof-of-principle studies of the present invention, by contrast, establish that use of a CD40:CD154 binding interrupter, alone or in combination with another immunomodulating or immunosuppressing agent (such as a CD28 signaling interrupter) promotes long-term, rejection free integration of heterologous (MHC-mismatched) donor tissue into a primate recipient. It is encouraging that the therapy disclosed herein was remarkably simple, involving the administration of therapeutic agents through a standard peripheral intravenous catheter, and was tolerated remarkably well by the recipients. This is in stark contrast to other regimens used to achieve lasting graft acceptance in primates, requiring ionizing radiation, administration of donor-derived bone marrow, and significant preoperative immunosuppression. The animals treated in studies described herein displayed no evidence of T cell activation or the cytokine release typically observed following treatment with antibodies directed at CD3, and prolonged survival has not carried with it a demonstrable cost in terms of opportunistic infection. In addition, no alterations in peripheral. blood hematological parameters were noted during these studies. Long-term survival was achieved without apparent clearing or global reductions in any lymphocyte subset, and without loss of in vitro T cell responsiveness. It is therefore unlikely that the observed effect is attributable to T cell destruction following antibody or fusion protein opsonization. The results are striking. Such success in outbred rhesus monkeys suggests that allograft (or even xenograft) integration is an achievable goal in humans, using this or an equivalent therapeutic approach.

The mechanism and relative contribution of each agent in the optional combination therapy described below remains unclear. The success of CD40:CD154 blockade alone suggests that any basal costimulation signaling is less important in maintaining the rejection response than CD80/CD86 upregulation. Indeed, anti-CD154 antibody administration resulted in impressive rejection-free survival when used alone, whereas the effects of the CD28 interrupter (the CTLA4-Ig) were more transient. Given that CD154 is expressed on non-myeloid cells, including vascular endothelium and smooth muscle, and that CD80 can be induced on fibroblasts and hepatocytes, non-T cell events may be critical in establishing reactivity against the donor tissue. By denying the immune system access to significant parenchymal adhesion and costimulatory signals at the time of transplantation, graft recognition and destruction may be prevented. The differences in activation induced by donor parenchyma and activation induced-by lymphoid cells could explain the observed preservation of in vitro reactivity to donor lymphocytes despite normal graft function, and the general poor correlation between MLR reactivity and-clinical graft outcome. Nonetheless, the effects of the exemplary costimulation blocking agents, CTLA4-Ig and humanized 5c8 (anti-human CD154), were shown to be synergistic both in vitro and in vivo. Perhaps, CTLA4-Ig provides insurance against CD80/CD86 expression that escapes the effects of CD40:CD154 binding interruption by humanized 5c8. In that instance, considerable time seems to be required to mount an effective-acute rejection with the few cells that escape initial blockade.

As this strategy was successful in reversing established, biopsy proven rejection, it would appear that the rejection process must be maintained by continuous costimulation, rather than being a process that, once set into motion, proceeds unless the effector cells are eliminated or rendered incapable of TCR signaling. Teleologically, the body is best served by inflammation that is easily controlled. Thus, in the absence of direction to attack, retreat may be the tacit order. This supports the view that exploitation of the immune system's natural propensity to down-regulate should be more advantageous than pan-immunosuppression.

The following discussion illustrates and exemplifies the variety of contexts and circumstances in which the invention can be practiced, as well as providing proof-of-principle studies involving specific embodiments of the invention.

Recipient Hosts

The invention can be used for treatment or prophylaxis of any mammalian recipient of a tissue graft or any mammal in need of a tissue graft. Preferably, the recipient (also referred to herein as the recipient host, or simply the host) is a primate, more preferably a higher primate, most preferably a human. In other embodiments, the recipient may be another mammal in need of a tissue graft, particularly a mammal of commercial importance, or a companion animal or other animal of value, such as a member of an endangered species. Thus, recipient hosts also include, but are not limited to, sheep, horses, cattle, goats, pigs, dogs, cats, rabbits, guinea pigs, hamsters, gerbils, rats and mice.

Donor or Graft Tissue

The invention can be used with any type of tissue transplant or graft procedure, particularly procedures wherein the donor (grafted) tissue is affected by, or at risk of, failure or rejection by the recipient host's immune system. In particular, the invention can be used in any context wherein the donor tissue is not histocompatible with the recipient host.

Thus, in addition to autologous or syngeneic donor tissue, the invention can be used with allogeneic or even xenogeneic donor tissue. The donor tissue can be derived, by conventional means, from a volunteer or other living donor, or from a cadaveric donor. The donor tissue may also be artificial tissue, such as artificial skin products. Preferably, the donor is as histocompatible as practicable with the recipient host. Thus, where the recipient host is a human, autologous and allogeneic donor tissue is preferred. However, the donor tissue can be obtained from a heterologous species (in which case it is referred to as a heterograft), such as a non-human primate (e.g., a chimpanzee or a baboon), or another relatively compatible mammal (e.g., a pig).

In some embodiments, the donor tissue comprises an organ, a portion of an organ, such as a liver, a kidney or a heart, or a body part comprising multiple tissue types such as a joint, a hand, a foot, a myocutaneous flap or a finger. In other embodiments, the donor tissue comprises a part, portion or biopsy of a donor organ or tissue. In still other embodiments, the donor tissue comprises cells, particularly isolated or suspended cells, including cells withdrawn or excised from a donor host, cells maintained in primary culture, or an immortalized cell line. Optionally, the donor tissue can include cells harboring exogenous genetic material, such as transfected or transformed host cells which have been (or are derived from ancestor cells which have been) engineered to include genetic material necessary for the production of a polypeptide of therapeutic value to the recipient host. In still other embodiments, the donor tissue can be derived from a transgehic mammal that has been engineered to include genetic material necessary for the production, in some or all of its body tissues, of a polypeptide of therapeutic value to the recipient host. Exemplary polypeptides of therapeutic value to the recipient include: hormones such as insulin or growth hormone; cytokines; growth and differentiation factors; enzymes; structural proteins; and the like.

In other embodiments, the donor tissue comprises synthetic (artificial), or biosynthetic (bioartificial) tissue, such as artificial replacement tissue for a variety of tissues, including skin and connective tissues. Several companies make or are making such products. These companies include, inter alia, Organogenesis, Inc. and Advanced Tissue Sciences, Inc. Engineered skin substitutes currently contemplated include, inter alia, those made of cultured human keratinocytes and fibroblasts attached to collagen-glycosaminoglycan or other collagen gel substrates (see, e.g., Boyce (1998), Med. Biol. Eng. Comput. 36(6): 791-800); see also, Auger et al. (1998) Med. Biol. Eng. Comput. 36(6): 801-12).

Thus, in light of the foregoing, it is clear that the invention can be used with such solid organ grafts as: transplanted kidney, liver, pancreas, lung, heart, and the like. Similarly, the invention can be used with sections or portions of the foregoing as well as with additional tissue types, especially renal, hepatic, pancreatic (particularly islet), respiratory, cardiac, skin, vascular, nerve, bone, bone marrow, cartilage, tendon, ligament, muscle, fat, mammary, gastrointestinal lining, epithelium, endothelium, connective tissue, and the like.

Furthermore, the invention can be used with body parts comprising multiple tissue types, such as for the replacement or other surgical alteration or reconstruction of an eye, ear, nose, digit (finger or toe), joint, blood vessel, nerve, muscle, limb, myocutaneous flap or other body parts.

The invention can also be used for reconstruction of complex wounds, such as those involving loss or degradation of some or all layers of the skin, optionally involving underlying connective tissues, musculature and the like.

In other embodiments, the invention can be used with a cell preparation or suspension, introduced systemically or locally into the recipient host. For example, isolated, suspended or dispersed cells can be infused intravascularly, or implanted into a desired site, such as a bone marrow cavity, the liver, within the kidney capsule or a joint capsule, intramuscularly, intraperitoneally, subcutaneously, intradermally or applied locally to a wound site. Exemplary cells include peripheral blood cells, bone marrow or any hematopoetic component thereof, mesenchymal stem cells, muscle satellite cells, hepatocytes, hormone-producing or neuroendocrine cells, fibroblasts, neural crest cells, endothelia, and the like. In some embodiments, the cells are mitotically competent and produce new tissue of donor origin. In other embodiments, the cells are not mitotically competent, but produce or express a polypeptide or other product of therapeutic value to the recipient.

It is also clear that the invention can be used with a skin graft procedure. The skin is a notoriously difficult tissue with which to achieve or maintain engraftment. Autografts are not always possible and there is therefore currently a great need for skin allografts and xenografts. A preferred route of administration for treating or inhibiting skin graft rejection is topical, subdermal, intradermal or subcutaneous, though systemic and other routes are also contemplated.

Another preferred route of administration includes direct application locally (by topical application, immersion or bath, or local injection) into the recipient tissue bed, or to the graft tissue itself. High local concentrations of the agent, particularly in areas of lymphatic drainage, are expected to be particularly advantageous.

The term skin tissue as used herein includes skin with all or some of its layers.

Exemplarv CD40:CD154 Interruptors

Therapeutic compounds useful for the methods of the invention include any compound that blocks the interaction of cell surface CD40 (e.g., on B cells) with CD40L (CD154) expressed on the surface of activated T cells. CD40:CD154 binding interrupter compounds, such as anti-CD40L compounds, that are specifically contemplated include polyclonal antibodies and monoclonal antibodies (mAbs), as well as antibody derivatives, such as chimeric molecules, conjugates of two or more monoclonal antibodies, preferably with the Fc portion removed (see, e.g., Ghetie et al., PCT application number PCT/US98/14222, filed Jul. 8, 1998, and Ghetie et al. Proc. Natl. Acad. Sci. 94:7509-14 (1997), the disclosures of both of which are incorporated by reference herein), humanized molecules, molecules with reduced effector functions, bispecific molecules, and conjugates of antibodies. In a preferred embodiment, the antibody is monoclonal antibody 5c8 (ATCC Accession No. HB 10916), as described in U.S. Pat. No. 5,474,771, the disclosure of which is hereby incorporated by reference. In a currently highly preferred embodiment, the antibody is a humanized 5c8. Other known antibodies against CD154 include antibodies ImxM90, ImxM91 and ImxM92 (obtained from Immunex), an anti-CD40L mAb commercially available from Ancell (clone 24-31, catalog # 353-020, Bayport, MN), and an anti-CD40L mAb commercially available from Genzyme (Cambridge, MA, catalog # 80-3703-01). Also commercially available is an anti-CD40L mAb from PharMingen (San Diego, catalog #33580D). Numerous additional anti-CD40L antibodies have been produced and characterized (see, e.g., WO 96/23071 of Bristol-Myers Squibb, the specification of which is hereby incorporated by reference). The choice of an appropriate anti-CD40L antibody will depend, in part, on whether the antibody specifically binds to the CD40L that is expressed on the surface of the recipient's activated T cells. For example, mAb 5c8 specifically binds to human CD40L, and also binds at least to CD40L of other primates such as rhesus monkey.

The invention also includes anti-CD40L molecules of other types, such as complete Fab fragments, F(ab′)2 compounds, VH regions, FV regions, single chain antibodies (see, e.g., WO 96/23071), polypeptides, fusion constructs of polypeptides, fusions of CD40 (such as CD40Ig, as in Hollenbaugh et al., J. Immunol. Meth. 188:1-7, 1995, which is hereby incorporated by reference), and small molecule compounds such as small semi-peptidic compounds or non-peptide compounds, all capable of blocking or interrupting CD40:CD154 binding. Procedures for designing, screening and optimizing small molecules are provided in the patent application PCT/US96/10664, filed Jun. 21, 1996, the specification of which is hereby incorporated by reference.

Various forms of antibodies may also be produced using standard recombinant DNA techniques (Winter and Milstein, Nature 349: 293-99, 1991). For example, “chimeric” antibodies may be constructed, in which the antigen binding domain from an animal antibody is linked to a human constant domain (an antibody derived initially from a nonhuman mammal in which recombinant DNA technology has been used to replace all or part of the hinge and constant regions of the heavy chain and/or the constant region of the light chain, with corresponding regions from a human immunoglobulin light chain or heavy chain) (see, e.g., Cabilly et al., U.S. Pat. No. 4,816,567; Morrison et al., Proc. Natl. Acad. Sci. 81:6851-55, 1984). Chimeric antibodies reduce the immunogenic responses elicited by animal antibodies when used in human clinical treatments.

Furthermore, conjugates of two or more monoclonal antibodies, preferably with the Fc portions removed, may also be used in the methods and composition of this invention. See, e.g., Ghetie et al., PCT application-number PCT/US98/14222, filed Jul. 8, 1998, and Ghetie et al. Proc. Natl. Acad. Sci. 94:7509-14 (1997), the disclosures of both of which are incorporated by reference herein.

In addition, recombinant “humanized” antibodies may be synthesized. Humanized antibodies are antibodies initially derived from a nonhuman mammal in which recombinant DNA technology has been used to substitute some or all of the amino acids not required for antigen binding with amino acids from corresponding regions of a human immunoglobulin light or heavy chain. That is, they are chimeras comprising mostly human immunoglobulin sequences into which the regions responsible for specific antigen-binding have been inserted (see, e.g., PCT. patent application WO 94/04679). Animals are immunized with the desired antigen, the corresponding antibodies are isolated and the portion of the variable region sequences responsible for specific antigen binding are removed. The animal-derived antigen binding regions are then cloned into the appropriate position of the human antibody genes in which the antigen binding regions have been deleted. Humanized antibodies minimize the use of heterologous (inter-species) sequences in antibodies for use in human therapies, and are less likely to elicit unwanted immune responses. Primatized antibodies can be produced similarly.

Another embodiment of the invention includes the use of human antibodies, which can be-produced in nonhuman animals, such as transgenic animals harboring one or more human immunoglobulin transgenes. Such animals may be used as a source for splenocytes for producing hybridomas, as described in U.S. Pat. No. 5,569,825.

Antibody fragments and univalent antibodies may also be used in the methods and compositions of this invention. Univalent antibodies comprise a heavy chain/light chain dimer bound to the Fc (or stem) region of a second heavy chain. “° Fab region” refers to those portions of the chains which are roughly equivalent, or analogous, to the sequences which comprise the Y branch portions of the heavy chain and to the light chain in its entirety, and which collectively (in aggregates) have been shown to exhibit antibody activity. A Fab protein includes aggregates of one heavy and one light chain (commonly known as Fab′), as well as tetramets which correspond to the two branch segments of the antibody Y, (commonly known as F(ab)2), whether any of the above are covalently or non-covalently aggregated, so long as the aggregation is capable of selectively reacting with a particular antigen or. antigen family.

In addition, standard recombinant DNA techniques can be used to alter the binding affinities of recombinant antibodies with their antigens by altering amino acid residues in the vicinity of the antigen binding sites. The antigen binding affinity of a humanized antibody may be increased by mutagenesis based on molecular modeling (Queen et al., Proc. Natl. Acad. Sci. 86:10029-33, 1989; PCT patent application WO 94/04679). It may be desirable to increase or to decrease the affinity of the antibodies for CD40L, depending on the targeted tissue type or the particular treatment schedule envisioned. This may be. done utilizing phage display technology (see, e.g., Winter et al., Ann. Rev. Immunol. 12:433-455, 1994; and Schier et al., J. Mol. Biol. 255:28-43, 1996, which are hereby incorporated by reference). For example, it may be advantageous to treat a patient with constant levels of antibodies with reduced affinity for CD40L for semi-prophylactic prophylactic treatments. Likewise, antibodies with increased affinity for CD40L and/or improved effector function may be advantageous for short-term treatments.

Routes of Administration

The compounds of the invention may be administered in any manner which is medically acceptable. Depending on the specific circumstances, local or systemic administration may be desirable. Preferably, the compound is administered via a parenteral route such as by an intravenous, intraarterial, subcutaneous, intramuscular, intraorbital, intraventricular, intraperitoneal, subcapsular, intracranial, topical, intraspinal, intradermal, subdermal or intranasal injection, infusion or inhalation route. The compound may also be administered via an oral or an enteral route. The compound also may be administered by implantation of an infusion pump, or a biocompatible or bioerodable sustained release implant, into the recipient host, either before or after implantation of donor tissue. Alternatively, certain compounds of the invention, or formulations thereof, may be appropriate for oral or enteral administration. Still other compounds of the-invention will be suitable for topical administration to asuitable tissue surface, such as a surgical site, a wound site (e.g. an abrasion or a burn) or any other tissue surface which permits uptake of the compound by the body of the recipient.

In general, compounds of the invention are administered to the recipient host. However, the compounds also can be administered to the donor, or to the donor tissue. For example, a compound of the invention can be included in a perfusion or preservative fluid in which the donor tissue is stored or transported prior to its integration into the recipient host. Alternatively, in the case of a graft comprising isolated or suspended cells, a compound of the invention can be included in the cell suspension, and the resulting mixture infused, e.g. intravenously.

For skin grafts, topical administration (including administration to graft beds and wound sites), subdermal application, including local injection, intradermal and subcutaneous application, and other methods that allow absorption of the compound into the graft bed are preferred routes of administering the anti-CD40L compound to a skin graft recipient; systemic administration is also possible.

For skin grafts and other grafts, where appropriate, the administration may be by means of an article of manufacture comprising an effective amount of a CD40:CD154 binding interrupter. The article of manufacture may be, inter alia, a dressing (e.g., a bandage) or an intradermal patch.

Dosages and Frequency of Treatment

The amount of and frequency of dosing for any particular compound to be administered to a patient for a given immune complex disease is within the skills and clinical judgement of ordinary practitioners of the tissue transplant arts, such as transplant surgeons. The general dosage and administration regime is established by preclinical and clinical trials, which involve extensive but routine studies to determine the optimal administration parameters of the compound. Even after such recommendations are made, the practitioner will often vary these dosages for different recipient hosts based on a variety of considerations, such as the individual's age, medical status, weight, sex, and concurrent treatment with. other pharmaceuticals. Determining the optimal dosage and administration regime for each anti-CD40L compound used to inhibit graft rejection is a routine matter for those of skill in the pharmaceutical and medical arts.

Generally, the frequency of dosing may be determined by an attending physician or similarly skilled practitioner, and might include periods of greater dosing frequency, such as at daily or weekly intervals, alternating with periods of less frequent dosing, such as at monthly or longer intervals.

To exemplify dosing considerations for an anti-CD40L compound, the following examples of administration strategies are given for an anti-CD40L mAb. The dosing amounts could easily be adjusted for other types of anti-CD40L compounds. In general, single dosages of between about 0.05 and about 70 mg/kg patient body weight are contemplated, with dosages most frequently in the 1-50 mg/kg range., particularly in the 1-20 range. For acute treatment, such as. before or at the time of transplantation, or in response to any evidence that graft rejection is beginning, an effective dose of antibodies is administered daily for a period of about 1 to 5 days, preferably by bolus intravenous administration. The same effective dosage, route and dosing schedule may be used in the load phase of a load-maintenance regimen, with the maintenance phase involving intravenous or intramuscular administration of antibodies for a treatment period of anywhere from weekly to 3 month intervals. Chronic treatment may also be carried out by a maintenance regimen, in which antibodies are administered by an intravenous or intramuscular route with interdose intervals ranging from about 1 week to about 3 months. In addition, chronic treatment may be effected by an intermittent bolus intravenous regimen, in which between about 1.0 mg/kg body weight and about 100 mg/kg body weight of antibodies are administered, with the interval between successive treatments being from 1 to 6 months. For all except the intermittent bolus regimen, administration may also be by oral, pulmonary, nasal, topical or subcutaneous routes.

According to an alternate embodiment of this invention for inhibition of graft rejection, the effectiveness of the antibodies may be increased by administration serially or in combination with conventional anti-rejection therapeutic agents or drugs such as, for example, corticosteroids or immunosuppressants. Alternatively, the antibodies may be conjugated to a conventional agent. This advantageously permits the administration of the conventional agent in an amount less than the conventional dosage, for example, less than about 50% of the conventional dosage, when the agent is administered as monotherapy. Accordingly, the occurrence of many side effects associated with that agent should be avoided.

Combination therapies according to this invention for treatment of graft rejection include the use of anti-CD40L antibodies together with agents targeted at B cells, such as anti-CD19, anti-CD28 or anti-CD20 antibody (unconjugated or radiolabeled), IL-14 antagonists, LJP394 (LaJolla Pharmaceuticals receptor blocker), IR-1116 (Takeda small molecule) and anti-Ig idiotype monoclonal antibodies. Alternatively, the combinations may include T cell/B cell targeted agents, such as CTLA4-Ig, cytokine antagonists such as IL-2 antagonists, IL-4 antagonists, IL-6 antagonists, and IL-15 antagonists, receptor antagonists, anti-CD80/CD86 and anti-B7 monoclonal antibodies, TNF antagonists, LFA1/ICAM antagonists, VLA4/VCAM antagonists,.LT/LTβ, CD2/LFA3 antagonists, brequinar and IL-2 toxin conjugates (e.g., DAB), prednisone, anti-CD3 mAb such as OKT3, mycophenolate mofetil (MMF), cyclophosphamide, CD45RB antagonists, rapamycin, and other immunosuppressants such as calcineurin signal. blockers, including without limitation, tacrolimus (FK506). Combinations. may also include T cell targeted agents, such as CD4 antagonists, CD2 antagonists and IL-12.

For maintenance of graft integration, or in a period following suppression of an acute episode of graft rejection, a maintenance dose of anti-CD40L antibodies, alone or in combination with a conventional anti-rejection agent is administered, if necessary. Subsequently, the dosage or the frequency of administration, or both, may be reduced. Where no sign of graft rejection is evident, treatment might cease, with vigilant monitoring for signs of graft rejection. In other instances, as determined by the ordinarily skilled practitioner, occasional treatment might be administered, for example at intervals of four weeks or more. Recipient hosts may, however, require intermittent treatment on a long-term basis upon any recurrence of disease symptoms.

Formulation

In general, compounds of the invention are suspended, dissolved or dispersed in a pharmaceutically acceptable carrier or excipient. The resulting therapeutic composition does not adversely affect the recipient's homeostasis, particularly electrolyte balance. Thus, an exemplary carrier comprises normal physiologic saline (0.15M NaC1, pH 7.0 to 7.4). Other acceptable carriers are well known in the art and are described, for example, in Remington's Pharmaceutical Sciences, Gennaro, ed., Mack Publishing Co., 1990. Acceptable carriers. can include biocompatible, inert or bioabsorbable salts, buffering agents, oligo-or polysaccharides, polymers, viscosity-improving agents, preservatives, and the like.

An anti-CD40L compound used in the methods of the invention is administered in a pharmaceutically-effective or therapeutically-effective amount, which is an amount sufficient to produce a detectable, preferably medically beneficial effect on a recipient host at risk or afflicted with graft. rejection. Medically beneficial effects would include preventing, delaying or attenuating deterioration of, or detectably improving, the recipient's medical condition. As an example, an indication of the status of a kidney allograft or xenograft, renal function and health may be monitored with one or more routine laboratory tests which measure the concentrations of relevant substances in blood or urine, other urine characteristics, or the rate of clearance of various substances from the blood into the urine. The parameters measured by these tests, either individually or in combination, can be used by a physician to assess renal function or damage. Examples of such parameters include the blood concentration of urea, creatinine or protein; the urine concentration of protein or of various blood cells such as erythrocytes or leucocytes; urine specific gravity; amount of urine; the clearance rates of inulin, creatinine, urea or Y-aminohippuric acid; and the presence of hypertension or edema.

As a specific example of a clinical use of the methods of the invention, in recipients of donor kidney tissue, anti-CD40L mAb (e.g., hu5c8) is administered perioperatively or to recipients presenting with evidence of graft rejection. Acute renal allograft rejection can be manifested by numerous indicia, including increases in serum creatinine or blood urea nitrogen, reduction in urine output, development of proteinuria and/or hematuria, or other indications of graft rejection. The amount and time course of immunomodulatory therapy should be sufficient to produce a clinically beneficial change in one or more of these indicia. An exemplary time course and dosage schedule is set forth in the proof-of-principle studies included herein. Essentially, however, the therapy involves administration of a CD40:CD154 binding interrupter (exemplified by hu5c8) intravenously as a bolus therapy in amounts up to 50 mg/kg, followed by an appropriate regime of subsequent administrations (e.g., daily intravenous or subcutaneous injections) for up to two weeks following initiation of therapy, or until evidence is obtained of the desired beneficial change in indicia of graft rejection or failure.

As another example, for recipients with evidence of other organ rejection, an anti-CD40L compound would be administered in a similar fashion as that described above. For example, acute rejection of liver transplants leads to jaundice (hyperbilirubuinemia), hepatitis (increased aminotransferase levels), coagulopathy and encephalopathy. Also, rejection of skin transplants leads to conditions such as exfoliative dermatitis and skin rash. Treating skin graft rejection by administering an anti-CD40L compound would benefit skin graft recipients such as burn victims, accident victims or recipients of skin reconstructive surgery.

As yet another example, in recipients of donor skin grafts, an anti-CD40L compound may be administered essentially as described above. Preferred administration routes are topical, subcutaneous, subdermal or intradermal, and local injection (in some instances, it might be advantageous to inject systemically). If preferred, the compound may be incorporated into a wound dressing such as a bandage or tissue adhesive film for closing a wound or surgical site. The donor skin may also be exposed (e.g. by immersion) to an anti-CD40L compound prior to transplantation.

Pre-Clinical Model Systems for Evaluating CD40:CD154 Interruptor Treatment Regimes

A preferred, exemplary model system for testing efficacy of a CD40:CD154 interrupting compound (e.g., an anti-CD40L compound, such as the monoclonal antibody 5c8) is the primate renal allograft model disclosed in prior related United States provisional application 60/049,389 and in Kirk et al. (1997), 94 Proc. Natl. Acad. Sci. U.S.A. 8789-8794, the teachings of both which are incorporated by reference herein. The present rhesus monkey model has been shown repeatedly to be a rigorous test of immune manipulation: one that is exquisitely sensitive to even minor changes in allograft function or adverse effects on recipient wound healing and immune system function. In addition, it has biological similarity to human renal transplantation. Specifically, genes that encode MHC proteins are well conserved between rhesus monkeys and humans, and their rejection of vascularized organs closely parallels that seen clinically.

It will be readily appreciated that this model system is suitable for evaluating grafts comprising renal (kidney) tissue. Other art-recognized preclinical model systems, preferably in primates, are suitable for assessing efficacy of other graft tissue types such as liver, heart, lung, pancreas, pancreatic islet, skin, peripheral or central nerve, or other tissue or organ types.

Materials and Methods Reagents

Human CTLA4-Ig and a control fusion protein-IgG1 were prepared as previously described and shipped in solution by Genetics Institute, Cambridge, MASS. The anti-CD40 ligand antibody, 5c8, was prepared as previously described (U.S. Pat. No. 5,474,771) humanized and shipped in solution by Biogen Corporation, Cambridge, MASS. The hamster anti-mouse CD28 monoclonal antibody PV-1 (IgG1, clone G62) was purified from hybridoma culture supernatants and used as an isotype control monoclonal antibody.

MHC Typing and Donor/Recipient Selection

Donor-recipient combinations and animals chosen for third party cells were selected based on genetic non-identity at both MHC class I and class II. Class I disparity was established by one-dimensional isoelectric focusing as previously described. Class II disparity was established based on the results of unidirectional mixed lymphocyte reactions (MLRs). In addition, the animal's DRB loci were verified to be disparate by denaturing gradient gel electrophoresis and direct sequencing of the second exon of DRB as previously described. Vigorous T cell responsiveness of the recipient towards the donor was confirmed in vitro for all donor-recipient pairs. The experiments described in this study were conducted according to the principles set forth in the “Guide for the Care and Use of Laboratory Animals” Institute of Laboratory Animals Resources, National Research Council, DHHS, Pub. No. NIH) 86-23(1985).

In Vitro Cellular Analvsis

Unidirectional MLRs were performed on all animals prior to transplantation and on rejection free survivors after 100 days. Each animal was tested against all potential donors to establish the highest responder pairs for transplantation. Responder cells (3×105) were incubated with irradiated stimulator cells (1×105) at 37° C. for 5 days. Cells were pulse-labeled with 3H-thymidine and proliferation was monitored by 3H-thymidine incorporation. Polyclonal stimulation with Concanavilin A (25 mcg/ml) served as a positive control. A stimulation index was calculated by normalization to self reactivity, which in all cases was near background incorporation. For in vitro dose response studies, CTLA4-Ig or humanized 5c8 was added to the MLR on day 1 at concentrations ranging from 100 mcg/ml to 0.01 mcg/ml. Combined treatments were performed by varying the CTLA4-Ig concentration and holding the humanized 5c8 concentration. steady at 50 mcg/ml.

Peripheral blood lymphocyte phenotype analysis was performed prior to transplantation and periodically during and after drug therapy. Assays. evaluated 0.2 ml of heparinized whole blood diluted with phosphate buffered saline and 1% fetal calf serum. FITC labeled T11, B1 (Coulter), and FN18 (the generous gift of Dr. David M. Neville, Jr.) monoclonal antibodies were used to assess the percentage of CD2 (T cell/NK cell), CD20 (B cell), and CD3 (T cell) positive cells respectively. Red blood cells were removed from the preparation by ACK lysis buffer (0.15 M NH4Cl, 1.0 mM KHCC3, 0.1 mM Na2EDTA, pH 7.3) treatment following staining. Cells were subjected to flow cytometry immediately, or following fixation in 1% paraformaldehyde. Flow cytometry was performed using a Becton Dickinson FACSCAN.

Renal Allografts

Renal allotransplantation was performed as previously described. Briefly, outbred juvenile (1 to 3 years of age) rhesus monkeys, seronegative for simian immunodeficiency virus, simian retrovirus, and herpes B virus, were obtained from the Primate Center (University of Wisconsin) or LABS (Yemassee, SC). Procedures were performed under general anesthesia using ketamine (1 mg/kg, i.m.), xylazine (1 mg/kg, i.m.) and halothane (1%, inhaled). Transplantation was performed between genetically distinct donor-recipient pairs as determined by the MHC analysis described above. The animals were heparinized during organ harvest and implantation (100 units/kg). The allograft was implanted using standard microvascular techniques to create an end to side an astamosis between the donor renal artery and recipient distal aorta as well as the donor renal vein and recipient vena cava. A primary ureteroneocystostomy was then created. Bilateral native nephrectomy was completed prior to closure.

Animals were treated with intravenous fluid for approximately 36 hours until oral intake was adequate. Trimethaprim-sulfa was administered for 3 days for surgical antibiotic prophylaxis. Each animal received 81 mg of aspirin on the day of surgery. The need for analgesia was assessed frequently and analgesia was maintained with intramuscular butorphanol. Animals were weighed weekly. Skin sutures were removed after 7 to 10 days. CTLA4-Ig and/or humanized 5c8 were given intravenously at doses and dosing schedules varying based on accumulating experience with the agents. No other immunopharmaceuticals were administered. Serum creatinine, and whole blood electrolytes (Na+, K+, Ca2+) and hemoglobin were determined every other day until stable and then weekly.

Pathological Analysis

Biopsies were performed on animals suspected of having rejection using a 20-gauge needle core device (Biopty-Cut, Bard). Standard staining with hematoxylin and eosin was performed on frozen or formalin fixed tissue to confirm the diagnosis of rejection. Animals were euthanized at the time of anuria or-if a weight loss of 15% of pre-transplant body weight occurred in accordance with AAALAC standards. All animals underwent complete gross and histopathological evaluation at the time of death.

Results

Both CTLA4-Ig and humanized 5c8 inhibited rhesus MLRs in a dose dependent fashion. CTLA4-Ig was, however, more effective than humanized 5c8as a single agent in preventing T cell proliferation. Substantial reduction in thymidine incorporation was seen at a CTLA4-Ig concentration of 0.1 mcg/ml, and further inhibition was achieved at higher concentrations. Modest reduction in proliferation was achieved with humanized 5c8 concentrations of 0.01 mcg/ml, but inhibition was not substantially improved by increasing concentrations. When tested in combination, both agents together inhibited proliferation approximately 100 times more effectively than did either agent alone. Dose response studies were repeated for 3 separate naive animals with identical results.

Twelve renal allotransplants were performed. Four animals received transplants without any immunological intervention. These animals rejected in 5, 7, 7 and 8 days respectively. Histological examination of their kidneys showed acute cellular rejection characterized by diffuse interstitial and tubular lymphocytic infiltration with edema and cellular necrosis. One animal was given a 5-day course of CTLA4-Ig (10 mg/kg/d) beginning at the time of transplantation and had graft survival prolonged to 20days. Graft loss was due to cellular rejection indistinguishable from that seen in the control animals. One animal was treated with CTLA4-Ig 20 mg/kg on the day of transplantation, followed by a 12 day course of 10 mg/kg every other day and had-graft survival prolonged to 30 days. Again, graft loss was due to acute cellular rejection. Extrapolating from previously published work in a rat heterotopic cardiac allograft model, a donor specific transfusion of lymph node derived lymphocytes (108) was given at the time of transplantation to these 2 animals.

Two animals were treated with humanized 5c8 alone. Both animals received 20 mg/kg every other day beginning on the day of surgery and continuing for 14 post-operative days (8 doses total). Both animals experienced extended rejection free survival, although transient creatinine elevations were recorded during the second and forth post-operative weeks. Both animals rejected between 95 and 100 days post-transplant. Biopsy was performed on each animal to confirm the diagnosis. Both animals were then re-treated with 7 doses of humanized 5c8 (20 mg/kg; one animal every other day and one animal daily) and both returned to normal graft function with no demonstrable adverse effects. They remained alive and well greater than 150 days after transplantation.

Two animals were given 20 mg/kg each of CTLA4-Ig and humanized 5c8 following transplantation. Again, each drug was given. every other day beginning on the day of surgery and continuing for 14 post-operative days. One animal rejected 32 days after surgery. The other remained free of rejection for 100 days, but like those animals treated with humanized 5c8 alone, rejected at that time. Similarly, a biopsy showed acute cellular rejection. The initial regimen of CTLA4-Ig and humanized 5c8 was repeated and the animal's creatinine level returned to baseline (1.0) MLR analysis following this treatment showed a donor specific loss of reactivity. Third party responsiveness was maintained. At 165 days post transplant, the animal was sacrificed as required by protocol due to weight loss. Graft function at that time was normal. At autopsy, the animal was found to have Shigella and Camphylobacter enterocolitis, a common infection in rhesus monkeys. This illness had infected multiple animals in the original primate colony, including several untreated animals. No other pathological abnormality was found; specifically, there was no evidence of lymphoproliferative disease or opportunistic infection. Histologically, the graft had isolated nests of lymphocytes in the interstitium, but no evidence of tubular infiltration, glomerular damage, or parenchymal necrosis.

Like the animals treated with humanized 5c8 alone, both of these animals had transient increases in their creatinine combined with an increase in graft size during the fourth post-operative week. It was hypothesized that this graft swelling reflected a second wave of infiltrating lymphocytes and therefore led to a modified dosage schedule such that both reagents were given on the day of surgery and on post-operative days 2, 4, 6, 8, 12, 16, and 28.

Two animals were treated with this modified regimen. Both have experienced rejection free survival, free of illness or alterations in renal functions for greater than 150 days. After 100 days of rejection free survival, MLRs were repeated against donor cells and third party cells. No changes in in vitro reactivity were observed. These studies were repeated after 150 days of rejection free survival with identical results. Both animals maintained vigorous in vitro responses toward donor and third party cells but failed to reject their allografts. No animal has demonstrated toxicity from any of the therapies employed. Specifically, there has been no fever, anorexia, or hemodynamic abnormalities, and no opportunistic infections have occurred. Animals have been housed in standard conditions and have been allowed contact with the other animals in the colony. They have maintained normal weight gain. Laboratory chemistries and hematological parameters such as hemoglobin and white blood cell counts have remained normal. The percentages of cells expressing CD2, CD3 and CD20 were unaffected by any treatment regimen. Specifically, no reduction in T cell counts was observed during or after treatment in any animal.

Further Pre-Clinical Studies using the Primate Renal Alloaraft Model System

The above-described primate renal allograft system was used subsequently to test various additional and/or further refined therapeutic regimes based on the use of humanized monoclonal antibody 5c8 as a monotherapy, or in combination with another therapeutic agent, e.g., CTLA4-Ig, MMF, tacrolimus, corticosteroids or a combination thereof.

### Monotheraphy for Renal Allograft in Primates

Two animals received monoclonal antibody 5c8monotherapy using an induction and maintenance regime as follows: The induction schedule involved administration of 20 mg/kg monoclonal antibody 5c8 at study days -1, 0, 3, 10 and 18, with day 0 being the day of renal allotransplantation surgery. Maintenance involved monthly administration of 20 mg/kg monoclonal antibody 5c8, beginning on study day 28. The treated animals remained essentially free of graft rejection, assessed by monitoring lymphocyte subset counts and/or serum creatinine level, as of study days 170 and 163, respectively.

Two additional animals received monoclonal antibody 5c8 monotherapy using a standard induction and low-dose maintenance regime as follows: The induction schedule involved administration of 20 mg/kg monoclonal antibody 5c8 at study days -1, 0, 3, 10 and 18, with day 0 being the day of renal allotransplantation surgery. Maintenance involved monthly administration of 10 mg/kg monoclonal antibody 5c8, beginning on study day 28. The treated animals remained essentially free of graft rejection as of study days 149 and 148, respectively.

Two further animals received monoclonal antibody 5c8 monotherapy using a low-dose induction and low-dose maintenance regime as follows: The induction schedule involved administration of 10 mg/kg monoclonal antibody 5c8 at study days -1, 0, 3, 10 and 18, with day 0 being the day of renal allotransplantation surgery. Maintenance involves monthly administration of 10 mg/kg monoclonal antibody 5c8, beginning on study day 28. The treated animals remain essentially free of graft rejection as of study days 38 and 9, respectively.

Yet two further animals received monoclonal antibody 5c8 monotherapy using a lower-dose induction and lower-dose maintenance regime as follows: The induction schedule involved administration of 5 mg/kg monoclonal antibody 5c8 at study days -1, 0, 3, 10 and 18, with day 0 being the day of renal allotransplantation surgery. Maintenance involves monthly administration of 5 mg/kg monoclonal antibody 5c8, beginning on study day 28. The treated animals rejected the renal implants at study days 7-10.

### Combination Therapies for Renal Allograft in Primates

All animals received monoclonal antibody 5c8 therapy using the standard 20 mg/kg induction and 20mg/kg maintenance regime described above, in combination with other immunosuppressive therapeutic regimes as follows: Three animals received combination therapy involving corticosteroids (e.g., methylprednisolone, using a 5 day induction course) and mycophenolate mofetil (MMF; 20 mg/kg po BID) at therapeutically effective doses. The treated animals remained essentially free of graft rejection as of study days 143, 81 and 80, respectively. In contrast, one control animal treated with similar doses of MMF and corticosteroids in the absence of monoclonal antibody 5c8 therapy rejected the renal implant at study day 7.

Two additional animals received combination therapy involving the immunosuppressant tacrolimus (formerly FK506) at therapeutically effective doses (1.5-2 mg/kg poBID, target trough 10 ng/ml). These treated animals remained essentially free of graft rejection as of study days 31 and 36, respectively.

Two further animals received combination therapy involving CTLA4-Ig at therapeutically effective doses. These treated animals remained essentially free of graft rejection at study days 122 and 3, respectively.

Further Pre-Clinical Studies Using the Primate Skin Allograft Model Svstem

The skin is a notoriously difficult tissue with which to achieve/maintain engraftment. Autografts are not always possible and there is therefore currently a need for skin allografts and xenografts.

Burn victims are in the greatest need for successful skin grafts. Other candidates include, for example, those requiring reconstructive surgery for birth defects or other conditions, patients suffering traumatic injuries (e.g., partial or complete amputation of a limb or other body parts) and those who need plastic surgery.

Favorable results of pre-clinical studies on primates involving administration of a CD40:CD154 interrupter (such as humanized 5c8 (“hu5c8”)) to skin graft recipients to inhibit or reverse graft rejection are discussed below.

Graft Donor and Recipient Animals

Nine primates (rhesus macaques) were used in the pre-clinical studies. The recipient animals were allogeneic to the donor animals. Graft donor/recipient pairs were assigned based on MLR high response and class II disparity determined by PCR analysis. Exemplary donor/recipient pairs were as follows: JB6/PC3; PC3/JB6, N9A/K4P and K4P/N9A.

The Day of Transplantation (Day 0)

Abdominal skin (full thickness) was taken from donor animals and defatted in normal saline with scissors and #10 blade. Abdominal skin wounds on the recipient animals were cleaned, and then ellipses of recipient skin were taken from the back at transverse axillary line. Both procedures were performed using aseptic technique. Skin grafts were placed on left scapula for autografts and right scapula for allografts.

Humanized monoclonal antibody (mAb) 5c8 (“hu5c8”) was administered intravenously at 20 mg/kg to each recipient prior to grafting. Additionally, hu5c8 (10 mgs) was administered beneath the graft by injection into each recipient's graft bed at the time of grafting.

Induction and maintenance therapy consisting of hu5c8 at 20 mg/kg was given as described above for renal transplant studies (e.g., on days 0, 3, 10, 18, 28 and then monthly).

Results

Skin grafts were examined on day 10 post-transplantation and daily thereafter.

A total of nine animals were transplanted to evaluate the feasibility of treating rejection of skin transplantation with hu5c8. Two animals failed the transplant procedure for technical reasons unrelated to rejection. Two animals received no anti-rejection therapy and rejected in 8-10 days as evidenced by dermatitis that progressed to graft necrosis. Five animals received hu5c8 as detailed above, intravenous injection and maintenance as well as local injection into the graft bed. One of these animals rejected at day 17 post-transplantation. Another one rejected at day 150 post-transplantation. Three animals remained well with functioning, well healed grafts as long as 200 days post-transplant. Thus, administration of hu5c8 as a sole therapy to prevent skin rejection significantly delayed the onset of acute skin graft rejection.

In ongoing transplant studies, donor specific transfusion (DST) has been utilized generally according to techniques described in U.S. Pat. No. 5,683,693. Administration of donor antigen (e.g. whole blood) with the CD40:CD154 binding interrupter may further reduce the incidence of graft rejection.

### Conclusion based on Preclinical Model Studies

The above-described results, taken together, indicate that induction of graft integration with the CD40:CD154 binding interrupter humanized 5c8 alone can lead to long-term survival of allografted tissue, including skin. The effects of humanized 5c8 can combine synergistically with the effects of a CD28 signaling interrupter, CTLA4-Ig, and are also compatible with several known immuno-suppressants and/or immunomodulatory agents.

### EOUIVALENTS

The invention may be embodied in other specific forms without departing from the spirit or essential characteristics thereof. The foregoing embodiments are therefore to be considered in all respects illustrative of, rather than limiting to, the invention disclosed herein. Scope of the invention thus is indicated by the appended claims rather than by the foregoing description, and all changes which come within the meaning and range of equivalency of the claims are intended to be embraced therein.

1. A method of treating or inhibiting rejection of a tissue graft by a primate graft recipient, comprising the step of administering an effective amount of a CD40:CD154 binding interruptor to said primate.

2. (canceled)

3. A method of reversing acute rejection of grafted tissue in a primate graft recipient, comprising the step of administering an effective amount of a CD40:CD154 binding interruptor to said primate.

4. A method of prolonging survival of grafted tissue in a primate graft recipient, comprising the step of administering an effective amount of a CD40:CD 154 binding interruptor to said primate.

5. A method of attenuating immunological complications of failure of grafted tissue in a primate graft recipient, comprising the step of administering an effective amount of a CD40:CD154 binding interrupter to said primate.

6. A method of delaying chronic rejection of a tissue graft by a primate graft recipient, comprising the step of administering an effective amount of a CD40:CD154 binding interruptor to said primate.

7. The method according to claim 1 further comprising the step of
implanting a tissue graft into said primate;
wherein the effective amount of a CD40:CD154 binding interruptor is administered to said primate with daily, weekly or monthly interdose intervals, counted from the day of implantation.

8. (canceled)

9. The method according to claim 1, wherein the step of administering an effective amount of a CD40:CD154 binding interrupter to a donor tissue is performed prior to transplanting said tissue to a primate graft recipient.

10. The method according to claim 1, further comprising the steps of:
(a) one day after administering an effective amount of a CD40:CD154 binding interruptor to a prospective primate graft recipient, implanting a tissue graft into said primate and concomitantly administering an effective amount of the CD40:CD154 binding interrupter to said primate; and
(b) administering effective amounts of the CD40:CD154 binding interrupter to said primate with daily, weekly or monthly interdose intervals, counted from the day of implantation.

11. (canceled)

12. The method according to claim 10, comprising an additional step of repeating administration of an effective amount of the CD40:CD154 binding interruptor to said primate, with interdose intervals ranging from 1 to 6 months, as counted from the day of implantation.

13. The method according to claim 3, wherein the effective amount of a CD40:CD154 binding interrupter is administered to said primate on the day on which said primate presents indicia of acute graft rejection, and with daily, weekly or monthly interdose intervals thereafter.

14. The method according to claim 13, comprising an additional step of repeating administration of an effective amount of the CD40:CD154 binding interrupter to said primate, with interdose intervals ranging from 1 to 6 months on a monthly, as counted from the day of presentation with indicia of acute graft rejection.

15. The method according to claim 1, wherein the CD40:CD154 binding interruptor is an anti-CD40L (anti-CD154) compound.

16. The method according to claim 15, wherein the anti-CD40L compound is a monoclonal antibody.

17. The method according to claim 15, wherein the anti-CD40L compound is an antibody derivative or an antigen-binding fragment of a monoclonal antibody.

18. The method according to claim 15, wherein the monoclonal antibody binds to the 5c8 antigen.

19. The method according to claim 18, wherein the monoclonal antibody has the antigen-specific binding characteristics of the 5c8 antibody produced by ATCC Accession No. HB 10916.

20. The method according to claim 1, wherein the grafted tissue is allogeneic or xenogeneic to said primate.

21. (canceled)

22. The method according to claim 1, wherein the grafted tissue comprises isolated or suspended cells.

23. The method according to claim 22, wherein said isolated or suspended cells are selected from the group consisting of:
(a) peripheral bloods cells; and
(b) bone marrow cells or any hematopoietic component thereof.

24. The method according to claim 1, wherein the grafted tissue is selected from the group consisting of renal, hepatic, cardiac, pancreatic, islet, skin, vascular, nerve, bone and cartilage tissue.

25. (canceled)

26. (canceled)

27. The method according to claim 1, wherein the grafted tissue is selected from the group consisting of an organ, a portion of an organ, and a body part comprising multiple tissue types.

28. The method according to claim 27, wherein the organ is heart, liver or kidney.

29. The method according to claim 27, wherein the body part is a myocutaneous flap, a joint, a hand, a foot or a finger.

30. The method according to claim 1, wherein the tissue comprises synthetic or biosynthetic tissue.

31. The method according to claim 30, wherein the biosynthetic tissue is bioartificial replacement tissue.

32. The method according to claim 30, wherein the bioartificial replacement tissue is bioartificial or artificial replacement skin tissue.

33. The method according to claim 1, comprising the additional step of administering an effective amount of an immunosuppressive or immunomodulatory compound to said primate.

34. The method according to claim 33, wherein the immunosuppressive or immunomodulatory compound is an agent that interrupts T cell costimulatory signaling via CD28.

35. The method according to claim 33, wherein the immunosuppressive or immunomodulatory compound is an agent that interrupts calcineurin signaling.

36. The method according to claim 35, wherein the agent is selected from the group consisting of cyclosporine and tacrolimus.

37. The method according to claim 33, wherein the immunosuppressive or immunomodulatory compound is selected from the group consisting of a corticosteroid and an antiproliferative agent.

38. The method according to claim 33, wherein the immunosuppressive or immunomodulatory compound is selected from the group consisting of sirolimus, mycophenolate mofetil, mizorubine, deoxyspergualin, brequinar sodium, leflunomide, azaspirane and rapamycin.

39. The method according to claim 1, wherein said primate is human.

40. The method according to claim 1, wherein the CD40:CD154 binding interrupter is administered to said primate graft recipient via a manner selected from the group consisting of:
(a) a parenteral route;
(b) a biocompatible or bioerodable sustained release implant;
and
(c) implantation of an infusion pump.

41. The method according to claim 40, wherein the CD40:CD154 binding interrupter is administered to said recipient by parenteral administration.

42. The method according to claim 41, wherein the CD40:CD154 binding interruptor is administered to said recipient by the parenteral administration selected from the group consisting of subcutaneous administration, intradermal administration, intramuscular administration, subdermal administration and topical administration.

43. The method according to claim 42, wherein the topical administration is by means selected from the group consisting of a dressing and an intradermal patch.

44. The method according to claim 1, wherein the CD40:CD154 binding interruptor is administered to a donor or graft tissue prior to integration of said tissue into said primate graft recipient.

45. The method according to claim 44, wherein the CD40:CD154 binding interrupter is administered to said donor or graft tissue by immersing that tissue in said interrupter.

46. A pharmaceutical composition comprising an anti-CD40L (anti-CD154) compound selected from the group consisting of a monoclonal antibody, an antigen-binding fragment thereof or an antibody derivative, wherein said compound has the antigen-specific binding characteristics of the 5c8 monoclonal antibody produced by ATCC Accession No. HB 10916, and an immunosuppressive or immunomodulatory compound selected from the group consisting of:
(a) an agent that interrupts T cell costimulatory signaling via CD28;
(b) an agent that interrupts calcineurin signaling;
(c) a corticosteroid; and
(d) an antiproliferative agent.

47. A pharmaceutical composition comprising an anti-CD40L (anti-CD154) compound selected from the group consisting of a monoclonal antibody, antigen-binding fragment thereof or an antibody derivative, wherein said compound has the antigen-specific binding characteristics of the 5c8 monoclonal antibody produced by ATCC Accession No. HB 10916, and an immunosuppressive or immunomodulatory compound selected from the group consisting of tacrolimus, sirolimus, mycophenolate mofetil, mizorubine, deoxyspergualin, brequinar sodium, leflunomide, and azaspirane.

48. A dressing for treating or inhibiting rejection of a tissue graft by a primate graft recipient, said dressing comprising an effective amount of a CD40:CD154 binding interruptor.

49. The dressing according to claim 48, wherein the dressing is a bandage.

50. The dressing according to claim 49, wherein the CD40:CD154 binding interruptor is an anti-CD40L (anti-CD154) compound.

51. The dressing according to claim 50, wherein the anti-CD40L compound is a monoclonal antibody.

52. The dressing according to claim 51, wherein the monoclonal antibody binds to the protein that is specifically recognized by monoclonal antibody 5c8 produced by ATCC Accession No. HB 10916.

53. The dressing according to claim 52, wherein the monoclonal antibody has the antigen-specific binding characteristics of the 5c8 antibody produced by ATCC Accession No. HB 10916.

54. An intradermal patch for treating or inhibiting rejection of a tissue graft by a primate graft recipient, said intradermal patch comprising an effective amount of a CD40:CD154 binding interruptor.

55. The intradermal patch according to claim 54, wherein the CD40:CD154 binding interruptor is an anti-CD40L (anti-CD154) compound.

56. The intradermal patch according to claim 55, wherein the anti-CD40L compound is a monoclonal antibody.

57. The intradermal patch according to claim 56, wherein the monoclonal antibody binds to the protein that is specifically recognized by monoclonal antibody 5c8 produced by ATCC Accession No. HB 10916.

58. The intradermal patch according to claim 56, wherein the monoclonal antibody has the antigen-specific binding characteristics of the 5c8 antibody produced by ATCC Accession No. HB 10916.
